(19) United States
(12) Patent Application Publication
(10) Pub. No.: US 2022/0172306 A1
(43) Pub. Date: Jun. 2, 2022

(54) AUTOMATED MOBILE FIELD SCOUTING SENSOR DATA AND IMAGE CLASSIFICATION DEVICES

(71) Applicant: BASF AGRO TRADEMARKS GMBH, Ludwigshafen am Rhein (DE)

(72) Inventors: Matthias TEMPEL, Leverkusen (DE); Marek Piotr SCHIKORA, Köln (DE)

(21) Appl. No.: 17/434,702

(22) PCT Filed: Feb. 28, 2020
(86) PCT No.: PCT/EP2020/055339

§ 371 Date: Aug. 27, 2021

(30) Foreign Application Priority Data
Feb. 28, 2019 (EP) 19160021.2

(51) Int. Cl. G06Q 50/02; G01N 21/3563; G01N 21/359; G01N 33/00; G06V 10/26; G06V 20/10

(52) U.S. Cl. CPC G06Q 50/02; G01N 21/3563; G01N 21/359; B64C 39/024; G06V 10/26; G06V 20/188; G01N 33/0098

(57) ABSTRACT

In order to provide a more effective field scouting management, a method is provided for field scouting management. The method comprises planning, using a mobile scouting management device with a display, a scouting trip in a plantation field, collecting scouting data along the planed scouting trip, providing the collected scouting data to a mobile decision-support device adapted for being carried into the plantation field, analyzing, using the mobile decision-support device, the scouting data to provide a scouting report comprising a field performance map, a weed map, a list of all identified species, and/S20 or a recommendation to re-scout, connecting the mobile decision-support device to the mobile scouting management device, and displaying the scouting report of the scouting trip on the display of the mobile scouting management device.

### FIELD OF THE INVENTION

This invention relates generally to field scouting, and more specifically to a method for field scouting management, to a mobile decision-support device, to a mobile scouting management device, and to a system for field scouting management.

### BACKGROUND OF THE INVENTION

Field scouting can be made more objective if a sensor carrier is deployed to collect field data, e.g. a drone. Analyzing the data can be automatized by image classification, but often requires a lot of computing power and takes very long to run on normal mobile consumer hardware. Although it is also possible to transmit the scouting data via an internet connection to a remote computing system for cloud computing, the data transmission may be time-consuming for a large field size, e.g. 600-1000 hectares, which may generate several terabytes (e.g. 20 TB or larger) of data. Furthermore, rural areas may have bad internet access for data transmission. This may provide a great challenge to review the results for classification quality or do additional/follow-up scouting activities to collect more information.

### SUMMARY OF THE INVENTION

There may be a need to provide a method and a device for a more effective field scouting management.

The object of the present invention is solved by the subject-matter of the independent claims. Further embodiments and advantages of the invention are incorporated in the dependent claims. The described embodiments similarly pertain to the method for field scouting management, to the mobile decision-support device, to the mobile scouting management device, and to the system for field scouting management.

A first aspect of the invention relates to a method for field scouting management. The method comprises planning, using a mobile scouting management device with a display, a scouting trip in a plantation field, collecting scouting data along the planed scouting trip, providing the collected scouting data to a mobile decision-support device adapted for being carried into the plantation field, analyzing, using the mobile decision-support device, the scouting data to provide a scouting report comprising a field performance map, a weed map, a list of all identified species, and/or a recommendation to re-scout, connecting the mobile decision-support device to the mobile scouting management device, and displaying the scouting report of the scouting trip on the display of the mobile scouting management device.

In other words, it is proposed to use a mobile decision-support device, e.g. a neural dongle device, instead of conventional cloud computing to process the scouting data. This may offer the advantages of processing scouting data directly in the field without the requirements of an internet connection (e.g. for cloud computing). This may be beneficial for a large field size, e.g. 600-1000 hectares, which may generate several terabytes (e.g. 20 TB or larger) of data, as it takes time to transmit the scouting data via the internet connection to the remote computing system for cloud computing. Processing scouting data directly in the field will save time and allow the farmer to review the results for classification quality or do additional/follow-up scouting activities to collect more information, e.g. a plant sample.

The term “mobile scouting management device” as used herein may include any type of wireless device such as consumer electronics devices, smart phones, tablet personal computers, wearable computing devices, personal digital assistants (PDAs), laptop computers, and/or any other like physical computing device that is able to connect to a communications network.

The term “mobile decision-support device”, also referred to as portable mobile decision-support device, as used herein may refer to a computing device small enough to hold and operate in the hand. The mobile decision-support device is connectable to the mobile scouting management device to provide it with additional more advanced computing functionality. Therefore, it is not required to provide the mobile decision-support device with a display or a user interface, such as a touchscreen interface with digital buttons or physical buttons along with a physical keyboard. Once the mobile decision-support device is connected to the mobile scouting management device, the user can use the display of the mobile scouting management device to view the scouting report and use the user interface of the mobile scouting management device to operate the mobile decision-support device. Additionally, the mobile decision-support device is capable of analysing the scouting data in the plantation field without need for an internet connection. The mobile decision-support device may be a small form factor device, which has a small size suitable for being carried in the field. A display is not required for the mobile decision-support device, as the results are made available to a user via the mobile scouting management device. The mobile decision-support device may be connected with the mobile scouting management device via a universal service bus (USB), a physical cable, Bluetooth, or another form of data connection to output the analysis result. As the mobile decision-support device is adapted to be carried in to the plantation field for processing the scouting data, the scouting data can be transferred to the mobile decision-support device via WLAN, SD-card or USB-cable to the mobile device without the requirement of an internet connection, such as cellular networks. Thus, the transmission of scouting data to the mobile decision-support device may be faster and more reliable.

According to an embodiment of the invention, analysing the collected scouting data further comprises using a pre-trained machine learned classifier to provide the scouting report based on the scouting data.

The pre-trained machine learned classifier may be trained using a plurality of labelled training data to recognize patterns, classify data, and forecast future events. The pre-trained machine learned classifier may be e.g. a decision tree, a support-vector machine, or an artificial neural network.

According to an embodiment of the invention, the scouting data comprise a plurality of captured georeferenced images of the plantation field. Analyzing the collected scouting data comprises stitching the plurality of georeferenced images together to obtain a stitched georeferenced image of the plantation field, calculating a field performance index based on the stitched georeferenced image to determine a measured value for vegetation at a plurality of locations and to provide the field performance map, comparing, at each of the plurality of locations, the measured value for vegetation and an expected value for vegetation coming from a crop modelling to determine a performance difference, and marking at least one point of interest for an additional data capture, where the determined performance difference is equal or above a reference value.

Optical remote sensing may be carried out, using satellite, drone, or radar platforms, to make use of e.g. visible, infrared (IR), near infrared (NIR), short-wave infrared, or multispectral sensors to form images of surface of the field by detecting the solar radiation reflected from targets on the ground. The vegetation parameter may be obtained by analysing the spectral signatures of the crop and soil in the image data. Examples of the field performance index are standardized precipitation index (SPI), vegetation optical depth (VOD), normalized difference vegetation index (NDVI), and/or enhanced vegetation index (EVI). The field performance index may bring together important information on various data points like plant height, soil type, soil moisture, and yield expectation. The ease and availability of data also means that the farmers can then quickly examines specific problem areas, i.e. the marked areas, allowing them to diagnose issues more.

According to an embodiment of the invention, analyzing the collected scouting data further comprises differentiating soil, crops and/or weeds area coverage, identifying at least one weed specie and providing a weed name of the at least one identified weed specie and a level of confidence indicative of a probability of a match between the weed name and the at least one identified weed specie, generating a list of the at least one identified weed specie with a high level of confidence, marking at least one weed with a low level of confidence for potential re-assessment; and grouping the at least one weed with a low level of confidence into an unidentified specie containing similar weeds.

The differentiation may be conducted based on the spectral signatures of soil, crops and weeds, that is, based on the fact that objects reflect and absorb various wavelengths in different amounts. Colors emerge because of the interaction between objects and light. Plants are special because light is an integral part of their lives. They have been found to absorb a lot of visible light, an energy source they use in photosynthesis. Conversely, their cell structure causes them to reflect a lot of NIR radiation. By comparing the reflectance of visual and NIR radiation, it can be determined which area in the field is covered by soil, crops, and weeds. The weed may be identified using their spectral signatures. Often, the spectral signatures of weeds common to an area or cropping system are available. This may allow the discrimination between weed and crop using spectral signatures and further the judgment of the percentage of weeds that contribute to the total cover of both crops and weeds in a given area, i.e. weed pressure. This may advantageously allow a better identification of locations infested with weeds and corresponding types for generating a weed map for later weed treatment.

According to an embodiment of the invention, the recommendation is provided for an additional field scouting, if the analysis result matches a predefined criterion. The predefined criterion comprises at least one of the following: an insufficient stitching quality of the stitched georeferenced image, an insufficient image quality of the stitched georeferenced image, at least one grouped unidentified weed specie, and at least one marked point of interest with a detected performed difference equal or above a reference value.

Insufficient image quality may be determined based on e.g. sharpness, color, artefacts, etc. These areas may represent specific problem areas. Therefore, the recommendation to schedule additional scouting activities in the field will allow the farmers to examine these specific problem areas to diagnose issues more.

According to an embodiment of the invention, the recommendation comprises a new map to re-scout with a sensor carrier based on the at least one marked point of interest and/or a map to do manual scouting for a user to verify the at least one unidentified specie and/or to investigate the at least one marked point of interest.

A second aspect of the invention relates to a mobile decision-support device. The mobile decision support device comprises a scouting data interface, a processing module, and a decision output interface. The scouting data interface is configured to receive scouting data of a plantation field. The processing module is configured to use a machine learned classifier to provide a scouting report based on the scouting data. The decision output interface is configured to output the scouting report.

In other words, the mobile decision-support device can be brought into the plantation field to process the scouting data without the need for an internet connection. A display is not required, as the scouting report can be output to be displayed on a mobile scouting management device. For example, the mobile decision-support device may be equipped with a special hardware and software that is designed to process the scouting data with artificial intelligence a magnitude faster than with consumer hardware. In one example, the mobile decision-support device may be a device that is pluggable into a port of a mobile scouting management device to provide the scouting report and to receive power supply. In another example, the mobile decision support device may be a stand-alone device with battery that is connectable to the mobile scouting management device wirelessly to provide the scouting report. In this way, all scouted can be process directly in the plantation field without the need for transmitting all scouting data to a remote server for cloud computing. This may allow a farmer to review the results immediately for classification quality or do additional or follow-up scouting activities to collect more information.

The term “interface” or “module” as used herein may refer to, be part of, or include an Application Specific Integrated Circuit (ASIC), an electronic circuit, a processor (shared, dedicated, or group) and/or memory (shared, dedicated, or group) that execute one or more software or firmware programs, a combinational logical circuit, and/or other suitable components that provide the described functionality.

According to an embodiment of the invention, the mobile decision-support device is a dongle device.

A dongle device is a small form factor computing device configured to be coupled to an external device, such as a mobile scouting management device. Typically, a dongle device includes a processor, storage, one or more data/power ports, and one or more wireless transceivers, but no power supply. That is, a dongle device must be plugged into an external device to supply power, thus differentiating it from a personal computer, laptop, tablet, mobile Internet, smart phone or other computing devices. Through a dongle device, a user may turn the mobile scouting management device into a fully-functioning computer for processing the scouting data. This may make it easy to be carried into the plantation field for processing the scouting data.

According to an embodiment of the invention, the mobile decision-support device further comprises at least one of the following: a battery unit for providing power to the device, a storage unit for storing the scouting data, and an indicator for indicating a battery life and/or a connectivity.

In other words, the mobile decision-support device may be a stand-alone hardware that is able to operate independently of other hardware or software, that is, the mobile decision-support device is not required to be plugged into an external device to supply power.

A third aspect of the invention relates to a mobile scouting management device. The mobile scouting management device comprises a user interface, a scouting planning module, a sensor carrier control interface, a decision input interface, and a display. The user interface is configured to receive a user input. The scouting planning module is configured to plan a scouting trip in a plantation field based on a user input and output the planned scouting trip to the sensor carrier control interface. The decision input interface is connectable to the decision output interface of the mobile decision-support device to receive a scouting report of the planned scouting trip. The display is configured to display the scouting report.

In other words, the mobile scouting management device is designed to plan, execute (i.e. steering of a sensor carrier), and provide a scouting report to the farmer, but not to process the scouting data. Rather, the scouting data is processed by the mobile decision-support device, which may be equipped with special hardware and software that is designed to process sensor data with artificial intelligence e.g. a magnitude faster than with consumer hardware. That is, the mobile decision-support device is provided to add more advanced computing functionality to the mobile scouting management device, which is usually a smartphone used by the farmer.

A fourth aspect of the invention relates to a system for field scouting management. The system comprises a mobile scouting management device as described above and below, a sensor carrier with a sensor carrier control interface, a sensor arrangement, and a scouting data interface, and a mobile decision-support device as described above and below. The mobile scouting management device is configured to allow a user to plan a scouting trip (26) in a plantation field. The sensor carrier control interface of the sensor carrier is connectable to the sensor carrier control interface of the mobile scouting management device to receive the planned scouting trip. The sensor arrangement is configured to collect scouting data at a plurality of locations along the scouting trip and to output the collected scouting data to the scouting data interface of the sensor carrier. The scouting data interface of the mobile decision-support device is connectable to the scouting data interface of the sensor carrier to receive the collected scouting data. The mobile decision-support device is configured to analyze the collected scouting data and to output a scouting report to the mobile scouting management device with a display for displaying the scouting report.

In other words, the system is designed to be automatically plan, execute and analyze the scouting data without the need for an internet connection. This may provide instant results to the farmers and allow, if needed, to schedule additional scouting activities in the field.

### BRIEF DESCRIPTION OF THE DRAWINGS

Exemplary embodiments of the invention will be described in the following with reference to the following drawings:

FIG. 1 shows a schematic drawing of a method according to an exemplary embodiment of the present disclosure.

FIG. 2 shows a schematic drawing of a method according to another exemplary embodiment of the present disclosure.

FIG. 3 shows a schematic drawing of a method according to a further exemplary embodiment of the present disclosure.

FIG. 4 shows a schematic drawing of a field according to an exemplary embodiment of the present disclosure.

FIG. 5 shows a schematic drawing of a mobile decision-support device according to an exemplary embodiment of the present disclosure.

FIG. 6 shows a schematic drawing of a mobile scouting management device according to an exemplary embodiment of the present disclosure.

FIG. 7 shows a schematic drawing of a system according to an exemplary embodiment of the present disclosure.

### DETAILED DESCRIPTION OF DRAWINGS

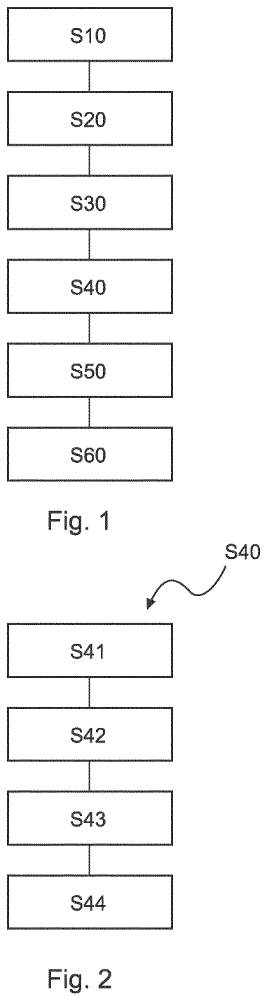

FIG. 1 shows a block diagram of an embodiment of a method for field scouting management. An example of a plantation field 10 is illustrated in FIG. 4. In step S10, a scouting trip is planned in the plantation field 10 using a mobile scouting management device 200 with a display 250. The mobile scouting management device 200 may be a smartphone as illustrated in FIG. 4. The mobile scouting management device 200 may has a mobile app or mobile application, which is used to plan the scouting trip, automatically or semi-automatically guide a sensor carrier across the field and present the results to the farmer.

In step S20, scouting data are collected along the planed scouting trip. The scouting data may be collected using satellite, drone, or radar platforms. For example, as illustrated in FIG. 4, to collect the scouting data, a sensor carrier 50 in form of a drone may be fitted with a sensor arrangement 70 with visual, IR, NIR, and/or thermal sensors.

In step S30, the collected scouting data are provided to a mobile decision-support device 100 adapted for being carried into the plantation field 10. The mobile decision-support device 100 may be equipped with special software that is designed to process sensor data with artificial intelligence faster than with consumer hardware. The scouting data may be transferred via WLAN, SD-card or USB-cable to the mobile decision-support device.

In step S40, the scouting data are analysed, using the mobile decision-support device 100, to provide a scouting report comprising a field performance map, a weed map, a list of all identified species, and/or a recommendation to re-scout. Optionally, the collected scouting data may be analysed using a pre-trained machine learned classifier to provide the scouting report of the scouting trip based on the scouting data. The pre-trained machine learned classifier may be a simpler version of a machine learned classifier in a high-performance remote server. Thus, the scouting data may be processed in a relatively fast speed.

In step S50, the mobile decision-support device 100 is connected to the mobile scouting management device 200 via a physical cable or wirelessly.

In S60, the scouting report is displayed on the display 250 of the mobile scouting management device 200.

In other words, the mobile scouting management device is designed to plan, to steer a sensor carrier and to display the scouting report, whereas the mobile decision-support device is designed to process the scouting data directly in the plantation field. The mobile decision-support device may have more computing power than consumer hardware and thus take less time to process the scouting data. Additionally, this may offer the advantages of processing scouting data directly in the field without the requirements of internet connection (e.g. for cloud computing). This may be beneficial for a large field size, e.g. 600-1000 hectares, which may generate several terabytes (e.g. 20 TB or larger) of data, as it takes time to transmit the scouting data via internet connection to the remote computing system for cloud computing.

Optionally, the scouting data comprise a plurality of captured georeferenced images of the plantation field. As illustrated in FIG. 2, analyzing S40 the collected scouting data comprises the following steps to determine additional points of interest detected by difference between an expected and measured performance. In step S41, the plurality of georeferenced images is stitched together to obtain a stitched georeferenced image of the plantation field.

In step S42, a field performance index, such as SPI, VOD, NDVI, and/or EVI, is calculated based on the stitched georeferenced image to determine a measured value for vegetation at a plurality of locations and to provide a field performance map. For example, as illustrated in FIG. 4, the plantation field 10 is divided into a plurality of grids in form of a rectangular array of squares 12a, 12b, 12c of equal size. The field performance map may be determined at the plurality of locations, e.g. at the plurality of squares 12a, 12b, 12c.

In step S43, the measured value for vegetation and an expected value for vegetation coming from a crop modelling are compared, at each of the plurality of locations, to determine a performance difference. For example, the performance difference at the plurality locations 12a, 12b, 12c may be calculated.

In step S44, at least one point of interest is marked for an additional data capture, where the determined performance difference is equal or above a reference value. The reference value may be set by the user or may be derived from the previous seasons. For example, the marked areas 12b may indicate the marked areas with the determined performance difference equal or above a reference value.

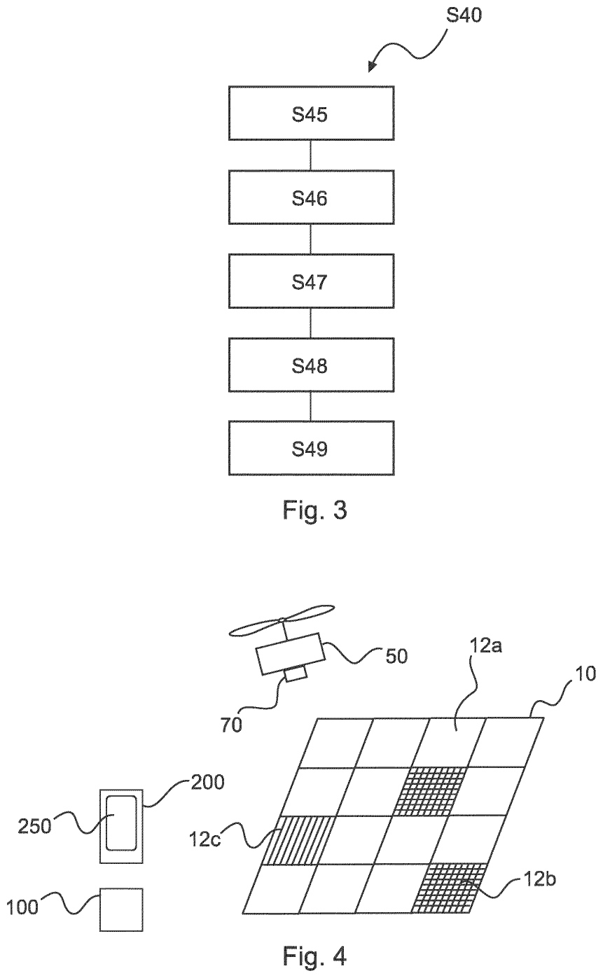

Optionally, as illustrated in FIG. 3, analyzing S40 the collected scouting data further comprises the following steps to determine identified and unidentified weed species. In step S45, in which a soil area coverage, a crop area coverage and a weed area coverage are differentiated. The differentiation may be conducted based on the spectral signatures of soil, crops and weeds, that is, based on the fact that objects reflect and absorb various wavelengths in different amounts.

In step S46, at least one weed specie is identified and a weed name of the at least one identified weed specie and a level of confidence indicative of a probability of a match between the weed name and the at least one identified weed specie is provided. Often, the spectral signatures of weeds common to an area or cropping system are available. This may allow the discrimination between weed and crop using spectral signatures and further the judgment of the percentage of weeds that contribute to the total cover of both crops and weeds in a given area, i.e. weed pressure.

In step S47, a list of the at least one identified weed specie with a high level of confidence is generated. The threshold for a high level confidence may be set by a user. The threshold of a high level of confidence may be set to be 50%, 60%, or 70%, etc.

In step S48, at least one weed with a low level of confidence is marked for potential re-assessment. For example, as illustrated in FIG. 4, the location 12c is marked for containing at least one weed with a low level confidence.

In step S49, the at least one weed with a low level of confidence is grouped into an unidentified specie containing similar weeds. In other words, similar unidentified weeds are grouped into one unidentified specie for the ease of weed management and treatment.

Optionally, the recommendation for an additional field scouting is provided, if the analysis result matches a predefined criterion. The predefined criterion comprises at least one of the following: an insufficient stitching quality of the stitched georeferenced image, an insufficient image quality of the georeferenced image or the stitched georeferenced image, at least one grouped unidentified weed specie, and at least one marked point of interest with a detected performance difference equal or above a threshold. For example, the marked locations 12b, 12c as illustrated in FIG. 4 may represent problem areas that requires further investigation and thus an additional field scouting. The recommendation may comprise at least one of the following: a new map to re-scout with a sensor carrier based on the at least one marked point of interest, and a map to do manual scouting for a user to verify the at least one unidentified specie and/or to investigate the at least one marked point of interest.

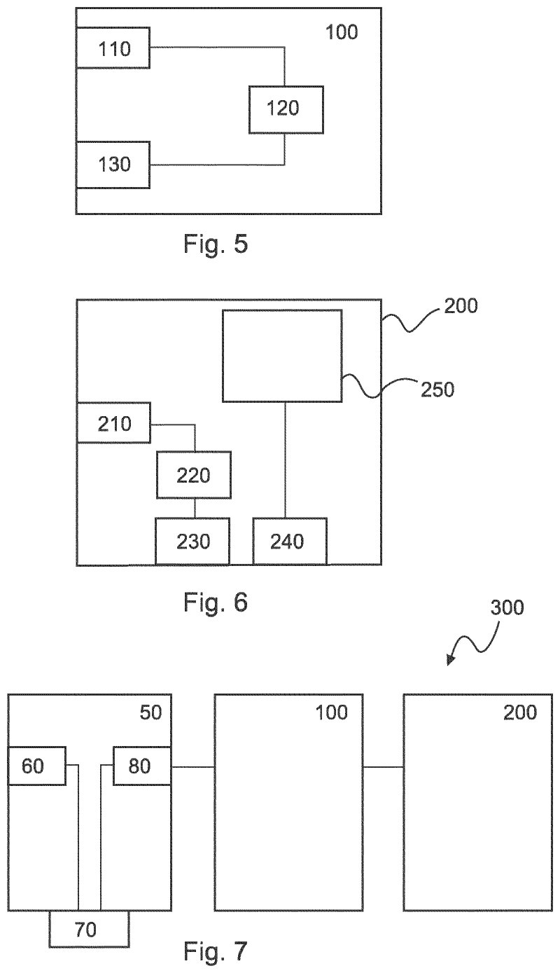

FIG. 5 schematically shows an embodiment of a mobile decision-support device 100. An example of the decision-support system 100 in form of a small form factor device is illustrated in FIG. 4. In an example, the mobile decision-support device 100 may be a dongle device that plugs into a mobile scouting management device 200, such as a smartphone, to add more advanced computing functionality. In another example, the mobile decision-support device 100 may be a stand-alone device with e.g. a battery unit for providing power to the device, a storage unit for storing the scouting data, and/or an indicator for indicating a battery life and/or a connectivity. In other words, the mobile decision-support device 100 may be a portable device connectable to the mobile scouting management device wirelessly to transmit the scouting report to the mobile scouting management device. The mobile decision-support device 100 comprises a scouting data interface 110, a processing module 120, and a decision output interface 130.

The scouting data interface 110 is configured to receive scouting data of a plantation field. The scouting data interface 110 may be a secure digital (SD) memory card interface, a universal serial bus (USB) interface, a Bluetooth interface, a wireless network interface, etc. suitable to receive the scouting data collected using satellite, radar or drone platforms. The scouting data may comprise radar image data or optical image data. The scouting data may also comprise GPS data adapted for providing locations of the identified problem areas.

The processing module 120 is configured to use a machine learned classifier to provide a scouting report based on the scouting data. The machine learned classifier may be a decision tree, a support-vector machine, an artificial neural network, etc. The scouting report may include a field performance map, a weed map, a list of all identified species, and/or a recommendation to re-scout.

The decision output interface 130 is configured to output the scouting report. The decision output interface 130 may be a USB interface, a Bluetooth interface, a wireless network interface, etc.

FIG. 6 schematically shows an embodiment of a mobile scouting management device 200, such as a smartphone as illustrated in FIG. 4 or a tablet computer. The mobile scouting management device 200 comprises a user interface 210, a scouting planning module 220, a sensor carrier control interface 230, a decision input interface 240, and a display 250.

The user interface 210 may be e.g. a pointing device, a keyboard, a touch panel, or another operation apparatus. The user interface 210 in form of a touch panel may also be integrated with the display 250.

The scouting planning module 220 is configured to plan a scouting trip in a plantation field based on a user input and output the planned scouting trip to the sensor carrier control interface 230.

The decision input interface 240 is connectable to the decision output interface 130 of the mobile decision-support device 100 to receive a scouting report of the planned scouting trip. The display 250 is configured to display the scouting report.

FIG. 7 schematically shows an embodiment of a system 300 for field scouting management. The system comprises a mobile scouting management device 200 as described above and below, a sensor carrier, a sensor carrier 50 with a sensor carrier control interface 60, a sensor arrangement 70, and a scouting data interface 80, and a mobile decision-support device 100 as described above and below.

The mobile scouting management device 200 is configured to allow a user to plan a scouting trip in a plantation field 10. The user may use the mobile scouting management device 200 to specify a plurality of GPS points to steer the sensor carrier 50 to collected data at these specified GPS points.

The sensor carrier control interface 60 of the sensor carrier 50 is connectable to the sensor carrier control interface 230 of the mobile scouting management device 200 to receive the planned scouting trip. The sensor arrangement 70 is configured to collect scouting data at a plurality of locations along the scouting trip and to output the collected scouting data to the scouting data interface 80 of the sensor carrier 50. The sensor carrier may include a fixed winged aircraft collecting data of the complete field, a multi rotor unmanned aerial system collecting data from predefined points of interest, and/or an unmanned ground vehicle collecting close proximity or sample data from point of interest where it is required. Scouting data can be transferred via WLAN, SD-card, or USB cable to the mobile decision-support device 100.

The mobile decision-support device 100 is configured to analyze the collected scouting data and to output a scouting report to the mobile scouting management device 200 with a display 250 for displaying the scouting report.

In another exemplary embodiment of the present invention, a computer program or a computer program element is provided that is characterized by being adapted to execute the method steps of the method according to one of the preceding embodiments, on an appropriate system. The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfil the procedure of an exemplary embodiment of the method as described above.

According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfil the functions of several items re-cited in the claims. The mere fact that certain measures are re-cited in mutually different dependent claims does not indicate that a combination of these measures cannot be used to advantage. Any reference signs in the claims should not be construed as limiting the scope.

### REFERENCE LIST

10 plantation field

12a location

12b point of interest

12c point of interest

50 sensor carrier

60 sensor carrier control interface

70 sensor arrangement

80 scouting data interface

100 mobile decision-support device

110 scouting data interface

120 processing module

130 decision output interface

200 mobile scouting management device

210 user interface

220 scouting planning module

230 sensor carrier control interface

240 decision input interface

250 display

S10 planning a scouting trip

S20 collecting scouting data

S30 providing the collected scouting data to a mobile decision-support device

S40 analyzing the scouting data

S41 stitching a plurality of georeferenced images

S42 calculating a field performance index

S43 comparing the measured value for vegetation and an expected value for vegetation

S44 marking at least one point of interest for an additional data capture

S45 differentiating a soil area coverage, a crop area coverage, and a weed area coverage

S46 identifying at least one weed specie

S47 generating a list of the at least one identified weed specie

S48 marking at least one weed

S49 grouping the at least one weed specie

S50 connecting the mobile decision-support device to the mobile scouting management device

S60 displaying the scouting report

1. A method for field scouting management, the method comprising:
planning (S10), using a mobile scouting management device with a display, a scouting trip in a plantation field (10);
collecting (S20) scouting data along the planed scouting trip;
providing (S30) the collected scouting data to a mobile decision-support device adapted for being carried into the plantation field;
analyzing (S40), using the mobile decision-support device, the scouting data to provide a scouting report comprising a field performance map, a weed map, a list of all identified species, and/or a recommendation to re-scout;
connecting (S50) the mobile decision-support device to the mobile scouting management device; and
displaying (S60) the scouting report on the display of the mobile scouting management device.

2. The method according to claim 1,
wherein analysing the collected scouting data further comprises using a pre-trained machine learned classifier to provide the scouting report based on the scouting data.

3. The method according to claim 1,
wherein the scouting data comprise a plurality of captured georeferenced images of the plantation field; and
wherein analyzing the collected scouting data comprises:
stitching (S41) the plurality of georeferenced images together to obtain a stitched georeferenced image of the plantation field;
calculating (S42) a field performance index based on the stitched georeferenced image to determine a measured value for vegetation at a plurality of locations and to provide a field performance map;
comparing (S43), at each of the plurality of locations (12a, 12b, 12c), the measured value for vegetation and an expected value for vegetation coming from a crop modelling to determine a performance difference; and
marking (S44) at least one point of interest (12b) for an additional data capture, where the determined performance difference is equal or above a reference value.

4. The method according to claim 1,
wherein analyzing the collected scouting data further comprises the following steps:
differentiating (S45) a soil area coverage, a crop area coverage and a weed area coverage;
identifying (S46) at least one weed specie and providing a weed name of the at least one identified weed specie and a level of confidence indicative of a probability of a match between the weed name and the at least one identified weed specie;
generating (S47) a list of the at least one identified weed specie with a high level of confidence;
marking (S48) at least one weed with a low level of confidence for potential re-assessment; and
grouping (S49) the at least one weed with a low level of confidence into an unidentified specie containing similar weeds.

5. The method according to claim 1, wherein the recommendation for an additional field scouting is provided, if the analysis result matches a predefined criterion, wherein the predefined criterion comprises at least one of the following:
an insufficient stitching quality of the stitched georeferenced image;
an insufficient image quality of the georeferenced image or the stitched georeferenced image;
at least one grouped unidentified weed specie; and
at least one marked point of interest with a detected performance difference equal or above a threshold.

6. The method according to claim 1, wherein the recommendation comprises at least one of the following:
a new map to re-scout with a sensor carrier based on the at least one marked point of interest; and
a map to do manual scouting for a user to verify the at least one unidentified specie and/or to investigate the at least one marked point of interest.

7. A mobile decision-support device (100), comprising:
a scouting data interface (110);
a processing module (120); and
a decision output interface (130);
wherein the scouting data interface is configured to receive scouting data of a plantation field;
wherein the processing module is configured to use a machine learned classifier to provide a scouting report based on the scouting data; and
wherein the decision output interface is configured to output the scouting report.

8. The device according to claim 7, wherein the mobile decision-support device is a dongle device.

9. The device according to claim 7, further comprising at least one of the following:
a battery unit for providing power to the device;
a storage unit for storing the scouting data; and
an indicator for indicating a battery life and/or a connectivity.

10. A mobile scouting management device (200), comprising:
a user interface (210);
a scouting planning module (220);
a sensor carrier control interface (230);
a decision input interface (240);
a display (250);
wherein the user interface is configured to receive a user input;
wherein the scouting planning module is configured to plan a scouting trip in a plantation field based on a user input and output the planned scouting trip to the sensor carrier control interface;
wherein the decision input interface is connectable to the decision output interface of the mobile decision-support device to receive a scouting report of the planned scouting trip; and
wherein the display is configured to display the scouting report.

11. A system (300) for field scouting management, comprising:
a mobile scouting management device (200) according to claim 10;
a sensor carrier (50) with a sensor carrier control interface (60), a sensor arrangement (70), and a scouting data interface (80); and
a mobile decision-support device (100);
wherein the mobile scouting management device is configured to allow a user to plan a scouting trip in a plantation field;
wherein the sensor carrier control interface of the sensor carrier is connectable to the sensor carrier control interface of the mobile scouting management device to receive the planned scouting trip;
wherein the sensor arrangement is configured to collect scouting data at a plurality of locations along the scouting trip and to output the collected scouting data to the scouting data interface of the sensor carrier;
wherein the scouting data interface of the mobile decision-support device is connectable to the scouting data interface of the sensor carrier to receive the collected scouting data; and
wherein the mobile decision-support device is configured to analyze the collected scouting data and to output a scouting report to the mobile scouting management device with a display for displaying the scouting report.

12. (canceled)

13. A non-transitory computer readable medium having stored thereon instructions that, when executed by a computing device, cause the computing device to perform the method of claim 1.
